(19) United States
(12) Patent Application Publication
(10) Pub. No.: US 2023/0368436 A1
(43) Pub. Date: Nov. 16, 2023

(54) SYSTEMS AND METHODS FOR COMPUTER VISION-ASSISTED COLORIMETRIC TEST READING

(71) Applicant: EMED LABS, LLC, Miami, FL (US)

(72) Inventors: Zachary Carl Nienstedt, Arden, NC (US); Tatiana Souslova, Tampa, FL (US)

(21) Appl. No.: 18/317,069

(22) Filed: May 13, 2023

Related U.S. Application Data
(60) Provisional application No. 63/341,947, filed on May 13, 2022.

(51) Int. Cl. G06T 11/00; A61B 5/00

(52) U.S. Cl. CPC G06T 11/001; A61B 5/7465; G06T 2210/41

(57) ABSTRACT

In some instances, the system may be used as part of a telehealth proctored examination, wherein a user is connected with a remote proctor via an electronic video conferencing link over an electronic network in order for the remote proctor to monitor, in real time, the examination and diagnostic process to ensure that the exam is being performed correctly and/or that the user is not cheating the exam while taking the exam or interpreting the results of the exam. During such telehealth proctored examination, the system can be configured to use the color gradient remapping features and processes disclosed herein to assist the user in interpreting and/or determining the results of an examination by allowing the user to more easily identify where a color of an exam result lies within a color gradient reference chart by remapping the color of the exam result and the colors of the gradient reference chart.

### TECHNICAL FIELD

The present application is directed to remote medical diagnostic testing and testing platforms. In some embodiments, the present application is directed to systems, methods and devices that are configured to utilize computer vision to read and/or interpret colorimetric testing results.

### BACKGROUND

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance patient and health provider contact, care, advice, reminders, education, intervention, monitoring, and remote admissions. Often, telehealth can involve the use of a user or patient's personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, and connecting testing users with medical providers who are needed to provide guidance and/or oversight of the testing procedures remotely.

More specifically, self-administered or at-home medical diagnostic tests often require a patient to compare a color on a test strip against a gradient of colors on a reference card in order to determine whether their test results are within an acceptable range. However, this can be difficult to accurately perform because the patient will often be forced to distinguish between various shades of a similar color. Thus, there is a growing need to improve the experience associated with self-administered or at-home medical diagnostic tests so that patients can quickly and more accurately determine the results of their medical diagnostics tests.

### SUMMARY

A user may administer a medical diagnostic test that may use colorimetry to indicate a presence, amount, or concentration of a substance of interest within a sample. The user may access a computer vision system through a website or application through use of a user device. The user can place a reference card and test strip of the diagnostic test within view of the user device camera to capture an image of the reference card and test strip in a single-frame view to be sent to the system. The test strip will have a test color associated with the result of the self-diagnostic test. The reference card will have a gradient of colors displayed on it. The reference card will also display one or more reference colors.

The system may receive the image and identify the reference card and test strip. The system can map a gradient of the reference card to a modified high-contrast gradient. The system may then display the modified gradient to the user through the user device. Either the user and/or the system can select a test result number that is associated with the color on the modified gradient that is most similar to the color that appears on the test strip. Based on the test result number selected, the system may direct the user accordingly.

In some embodiments, a user's mobile device may be used to capture an image of both the test strip and the reference card in a single-frame view. The test strip will have a test color associated with the result of the self-diagnostic test. The reference card will have a gradient of colors displayed on it. The reference card will also display one or more reference colors. A color correction may be applied to the image based on the one or more reference colors of the reference card that is captured in the image, by comparing those observed reference colors to the known baseline for what those reference colors should be.

Based on the color correction, a color-corrected gradient is obtained from the gradient of colors and a color-corrected test color is obtained from the test color. A transfer function is then used to remap the color-corrected gradient into a remapped high-visibility color gradient, and also to remap the color-corrected test color into a remapped test color based on the remapped high-visibility color gradient. The resulting remapped gradient and remapped test color will improve a person's ability to determine where the test color lies within the gradient.

The remapped test color and remapped gradient can then both be displayed to the patient on his or her mobile device so that the patient can make a comparison. One way to display this information in the display of the mobile device is via augmented reality by directly overlaying the remapped test color over the test color of the test strip and overlaying the remapped gradient over the gradient on the reference card, so that the patient can laterally slide the test strip along the gradient on the reference card while observing and comparing the remapped colors on the mobile device's display in real time. Another way to display this information in the display of the mobile device is to generate a processed still image that includes the remapped gradient adjacent to an elongated strip of the remapped test color that spans the length of the remapped gradient, which would allow the patient to more easily determine where in the remapped gradient the remapped test color lies.

For purposes of this summary, certain aspects, advantages, and novel features of the invention are described herein. It is to be understood that not necessarily all such advantages may be achieved in accordance with the particular embodiments of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

These and other features, aspects, and advantages of the present application are described with reference to drawings of certain embodiments, which are intended to illustrate but not limit the present disclosure. It is to be understood that the attached drawings are for the purpose of illustrating concepts disclosed in the present application and may not be to scale;

FIG. 1 illustrates an example embodiment of a gradient of a reference card and a high-contrast gradient used to interpret test results;

FIG. 2A illustrates an example embodiment of a test reference card;

FIG. 2B illustrates an example embodiment of a test strip;

FIG. 3A illustrates an example embodiment of a user device capturing an image of a reference card and test strip in a single-frame view.

FIG. 3B illustrates an example embodiment of a user device displaying an augmented reality version of the test using a high-contrast gradient.

FIG. 3C illustrates an example embodiment of a user device displaying a still image of the test using a high-contrast gradient.

FIG. 4 is a block diagram illustrating an example protocol or method for a system that implements computer vision for colorimetric test reading.

FIG. 5 is a block diagram illustrating an embodiment of a computer hardware system configured to run software for implementing one or more embodiments of the health testing and diagnostic systems, methods, and devices disclosed herein.

FIG. 6A is a flow diagram illustrating how color correction and gradient remapping can be performed in accordance with an example embodiment.

FIG. 6B illustrates how color correction and gradient remapping can be performed in accordance with FIG. 6A.

### DETAILED DESCRIPTION

Although several embodiments, examples, and illustrations are disclosed below, it will be understood by those of ordinary skill in the art that inventions described herein extend beyond the specifically disclosed embodiments, examples, and illustrations and include other uses of inventions, obvious modifications, and equivalents thereof. Embodiments of the inventions are described with reference to accompanying figures, wherein like numerals refer to the like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

As mentioned briefly above and as will now be explained in more detail below with reference to the example embodiments provided in the figures, this application describes systems, methods, and devices that are configured to utilize computer vision to read and/or interpret colorimetric testing results, and/or to remap colors of a colored reference chart and/or the color of test strip and/or test pad. In some embodiments, the systems disclosed herein can be configured to automatically and/or dynamically analyze an image and/or video frame of a reference chart and/or a test strip, and apply a model developed based on machine learning to determine and/or select the best color mapping schema in order to dynamically generate for the user with a new image and/or augmented reality image based on a color remapping schema that helps the user to interpret the test strip and/or determine a level, threshold, section where the test strip falls within a color reference chart. In some embodiments, the foregoing model is based on a data set of various color reference charts and/or test strips and/or test strip pads, wherein such data is used for training-and-evaluation iterations for generating the machine learning model. For example, a first training set of the data set is used to train a machine learning model during a first training-and-evaluation iteration. A second data set comprising ground truths is used to evaluate the generated machine learning model during a second training-and-evaluation iteration. In some embodiments, this process can be repeated to improve the generated machine learning model in order to improve the model's ability to select the most appropriate color remapping schema for any presented color reference chart, test strip, and/or test pad indicator. In some embodiments, the generated machine learning model can be configured to predict and/or select one or more color remapping schemas from a plurality of color remapping schemas that will likely create starker differences between color gradients within a color reference chart that would be best applicable to the color of the test strip, test pad indicator, or the like. In some embodiments, the use of machine learning techniques in the systems disclosed herein can include the use of deep learning and neural networks to develop more complex models that can capture non-linear relationships between the input data (for example, a plurality of color reference charts and/or color test strips and/or test indicator pads) and the predicted outcomes (for example, the selection of the most optimal color remapping schema). In some embodiments, the generated machine learning model comprises a mathematical equation and/or a set of rules that correlate the input data to the predicted outcomes. In some embodiments, the machine learning model is configured to comprise a feature selection step to identify the most relevant features in the dataset (for example, a plurality of color reference charts and/or known reference colors positioned adjacent or near the color reference chart, and/or color test strips and/or test indicator pads) that contribute to the prediction. In some embodiments, the feature selection step involves ranking the features based on their importance and selecting only the top features to improve the model's performance. Embodiments of the inventions described herein can comprise several novel features and no single feature is solely responsible for the desirable attributes or is essential to practicing the inventions described.

For example, in some instances, a user may administer a medical exam, health diagnostic, or the like. Such diagnostic or health test can include a diagnostic test for urinary tract infections (“UTI”) or other diagnostic tests that may use colorimetry to indicate a presence, amount, and/or concentration of a substance of interest within a sample, such as urine, saliva, blood, skin, cells, fluids, or the like. Such diagnostic test administered may include a test strip that has at least one indicator pad. Various tests may include multiple indicator pads on multiple or a single test strip, which may be sensitive to different or specific substances of interest. Each indicator pad may vary in chemical makeup such that interactions with specific substances of interest may cause each indicator to change to a different color. For example, a first indicator pad can turn a shade of yellow and a second indicator pad can turn a shade of violet.

In some instances, to administer the diagnostic test, the user may expose the indicator pad to the substance of interest within the sample to induce a chemical reaction within the indicator pad that may cause the indicator pad to change color. In some embodiments, the resulting color of the indicator pad may be referred to as a test color that is associated with the result of the diagnostic test. In some instances, the user can compare the color of the indicator pad to a reference card that may include a gradient or color block, in order to determine which color on the reference card is closest to the color of the indicator pad. In some instances, each area of the gradient can be mapped to a number scale (e.g., from 1-10). The area of the gradient that may match the user's indicator pad can correspond to a test result number. For example, the user's indicator pad can correspond to a test result number of 3.

In some instances, a system (such as a computer vision system) may be configured to read and/or interpret colorimetric test results. For example, after the user administers the diagnostic test, the user may open a website or application on a user device (such as a cell phone, smartphone, tablet, laptop, personal digital assistant (PDA) or the like). The user device may include a camera. Using the website or application, the user may place the reference card and the test strip within view of the user device camera such that all possible color outcomes and variations and the indicator pad within the test strip may be visible within a single image frame of the user device camera. The user may capture images of the reference card and test strip with the user device that may be received by the system through use of the website or application.

In some instances, the system can identify the reference card and the indicator pad within the test strip. The system can map the gradient of the reference card to a modified gradient. Such modified gradient can include a gradient that may have a higher color density than the gradient of the reference card. The modified gradient may include a single-color gradient that spans a full range of brightness values (i.e., from white or black) or a full spectrum of visible light colors (i.e., red, orange, yellow, green, blue, indigo, violet). For example, the left end of the gradient of the reference card may appear light yellow and appear deep red on the modified gradient. For example, the right end of the gradient of the reference card may appear a mid-range yellow and appear a deep blue on the modified gradient. The modified gradient can be customized to the user. For example, a color blindness test can be administered before generating the modified gradient to determine if the user is able to see the colors within the modified gradient. The modified gradient can be customized to omit any color or variation of color that the user may not be able to see or distinguish. By mapping the gradient of the reference card to the modified gradient of the user, the modified gradient can be displayed on the user's device.

In some instances, the system may detect the color of the indicator pad and map or otherwise associate the indicator pad with the associated color of the modified gradient. The indicator pad may appear as the associated color of the modified gradient on the user device. The system can display the color modified indicator pad and the modified gradient to the user on the user device. For example, the system may use augmented reality to display the color modified indicator pad and the modified gradient to the user on the user device.

In some instances, the user can compare the modified gradient and the color modified indicator pad displayed on the user device. Through the display, the user may see a greater color and/or brightness differentiation between adjacent colors within the modified gradient, compared to the gradient of the reference card.

In some instances, the system can map the modified gradient to a test result number. Such test result number can vary in range, for example, 1-10. The test result number may be identical to the test result numbers assigned on the reference card. This may allow the user to more easily determine the numerical test result. In some instances, the user may input his or her numerical test result into the system. The system may save the numerical test result to a user account associated with the user.

In some instances, the system can interpret the test results for the user. For example, the system may track the gradient of the reference card and the indicator pad and identify the color of the indicator pad (e.g., using an RGB color code). The system may further identify the area on the gradient of the reference card that the same or similar RGB color code is located. The system can read the nearest test result number associated with the color and provide the test result number to the user, which can indicate the diagnostic test result. Such indication can serve as an interpretation of the diagnostic test results by the system. Alternatively, the system may suggest a range of test result numbers in which the test result is likely to fall. This may provide the user with a narrower range of test result numbers to select from while still allowing the user to make the final determination and interpretation.

In some instances, once the test results are interpreted and input into the system, the system may trigger a test-to-treat process that can provide the user with a path to receive medication or other treatments for the detected problem.

In some instances, with the use of more than one indicator pad, each indicator pad may have its own results gradient, for example, a first results gradient with shades of yellow and a second results gradient with shades of violet. The system may identify and associate the first indicator pad with the first results gradient to assist with determining a first result. The system may identify and associate the second indicator pad with the second results gradient to assist with determining a second result. By stepping through the multiple test readings one at a time, the system may facilitate easier result readings for the user. In some instances where the system may interpret the diagnostic test result, each indicator pad can be read substantially simultaneously, which may allow for multiple results to be displayed to the user at once.

In some instances, the system may apply to food service sanitation tests for commercial kitchens, pool and spa water pH tests, urine analysis for hydration testing, etc.

In some instances, the system may be used as part of a telehealth proctored examination, wherein a user is connected with a remote proctor via an electronic video conferencing link over an electronic network in order for the remote proctor to monitor, in real time, the examination and diagnostic process to ensure that the exam is being performed correctly and that the user is not cheating on the exam while taking the exam or interpreting the results of the exam. During such telehealth proctored examination, the system can be configured to use the color gradient remapping features and processes disclosed herein to assist the user in interpreting and/or determining the results of an examination by allowing the user to more easily identify where a color of an exam result lies within a color gradient reference chart by remapping the color of the exam result and the colors of the gradient reference chart to incorporate starker color gradients in the reference chart, rather than typical gradual color gradients (for example, more different colors rather than just different shades of a single or a couple of colors).

Colorimetric test results can be difficult to read with accuracy and precision. Generally, possible color results may exist on a limited, single-color gradient (e.g., from very light yellow to a yellow of a mid-range brightness). This limitation can make it difficult for a user to determine which area of the results gradient matches the color of the user's indicator pad most closely. Simplifying test result interpretation for a user can contribute to more accurate and precise results of the medical diagnostic exam. Accordingly, it may be beneficial to provide a system that can utilize computer vision to read and/or interpret colorimetric testing results.

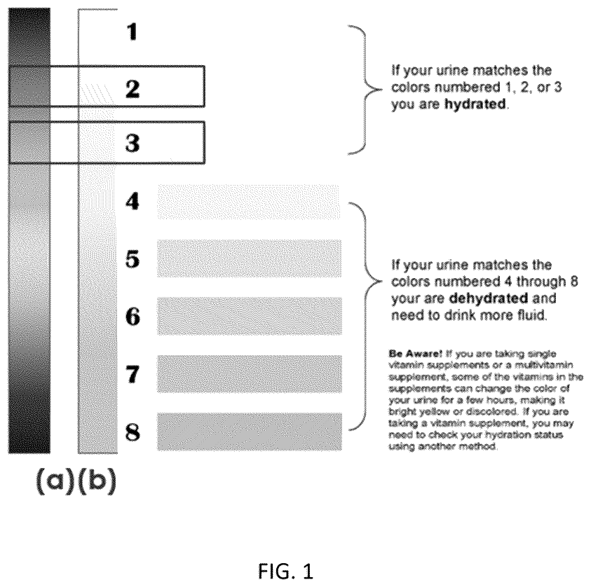

FIG. 1 illustrates an example embodiment of an original gradient and a high-contrast gradient used to interpret test results. For example, gradient (b) on the right can match the color guide for the color of urine and gradient (a) on the left can appear as an arbitrary high-contrast gradient. In some embodiments, an algorithm can take a color on gradient (b), map it to gradient (a), and display the corresponding color to the user. This may ease the interpretation of the color for the user, which can minimize error. In some embodiments, a user trying to distinguish between number 2 and number 3 on the yellow, or urine, color gradient may have a difficult time distinguishing between the colors. In some embodiments, a user trying to distinguish between number 2 and 3 on the high-contrast gradient can have less difficulty distinguishing between the colors. In some embodiments, the high-contrast gradient may be a high-visibility color gradient that improves a person's ability to determine where the test color lies within the gradient.

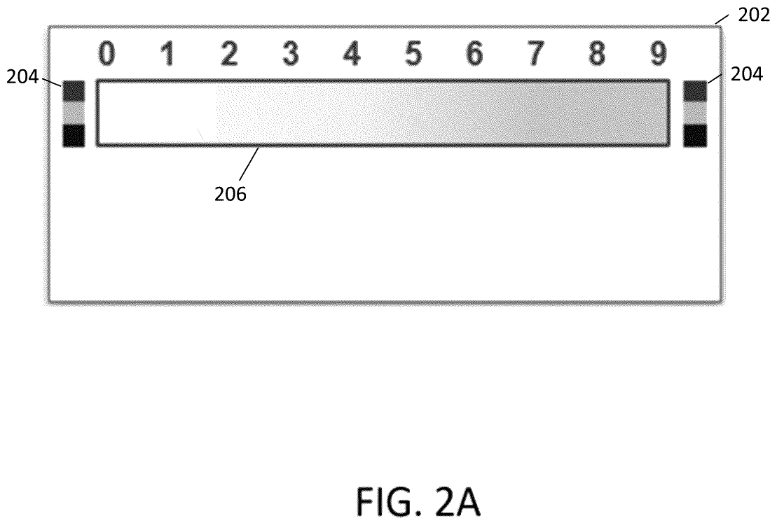

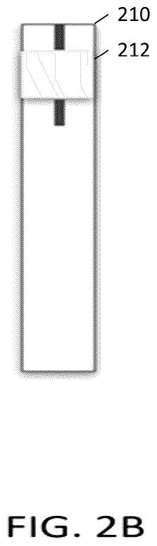

FIG. 2A illustrates an example embodiment of a test reference card. The test reference card 202 may be printed on the test box itself or it may be included as a separate print on a piece of cardstock. It may be used as a background and reference for results interpretation. The reference card 202 may include a gradient 206 of colors and one or more reference color areas 204. The reference color areas 204 may include one or more reference colors, and the reference colors may be chosen so that they do not conflict with the colors of the gradient 206.

FIG. 2B illustrates an example embodiment of a test strip. The test strip 210 may include an indicator pad 212 that can absorb the sample and appear as a color that may need to be interpreted.

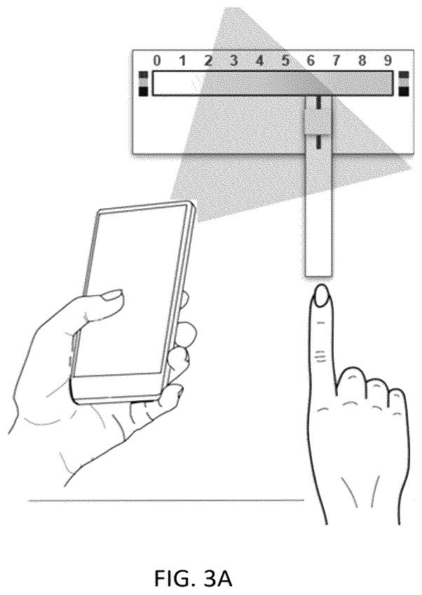

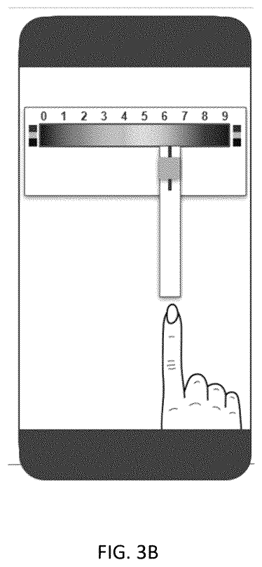

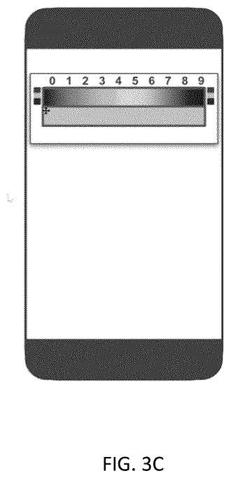

FIG. 3A illustrates an example embodiment of a user device capturing an image of a reference card and test strip in a single-frame view. The user may place the test strip on the test reference card and aim the user device camera at the test strip as shown in FIG. 3A. This may allow the user to see the test strip and the test reference card in the camera's view finder. Once this image is captured, the system may perform processing on the image (e.g., color correction and gradient remapping) before providing an updated test displayed on the user device, such as the augmented reality version of the test as depicted in FIG. 3B or the still image of the test as depicted in FIG. 3C.

FIG. 3B illustrates an example embodiment of a user device displaying an augmented reality version of the test using a high-contrast gradient. Through the use of gradient remapping, a remapped gradient can be overlaid and displayed over the gradient of the reference card while the remapped test color is overlaid and displayed over the test color of the test strip. When looking at the user device screen, the user may see an augmented reality version of the test as shown in FIG. 3B. The augmented reality version of the test may include substituted or modified colors for easier interpretation. The test displayed on the user device may be updated in real time, such that as the user slides the test strip along the reference card (within frame of the user device's camera), the overlaid remapped test color moves with the test strip to allow the user to determine where in the remapped gradient the remapped test color best fits.

FIG. 3C illustrates an example embodiment of a user device displaying a still image of the test using a high-contrast gradient. Through the use of gradient remapping, a still image can be generated that includes the remapped gradient adjacent to the remapped test color of the test strip. For better visibility, the remapped test color may span along a length of the remapped gradient, such as the entire length of the remapped gradient. This updated image can be displayed to the user on the user device in order to improve the user's ability to determine where the remapped test color lies within the remapped gradient.

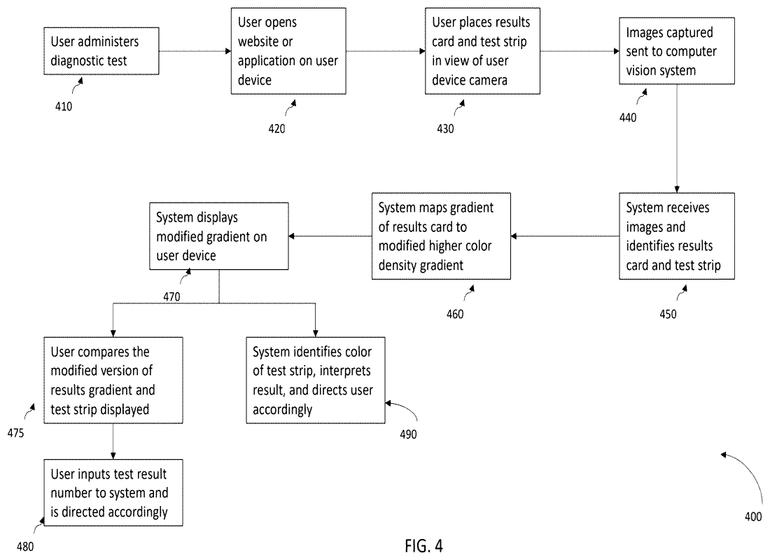

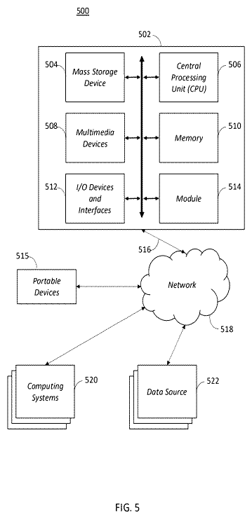

FIG. 4 illustrates a block diagram of an example protocol or method 400 for a system that implements computer vision for colorimetric test reading. The method 400 can be implemented, for example, using one or more components of the system shown in FIG. 5.

At block 410, a user may administer a diagnostic test. As described above, the diagnostic test can include a diagnostic test for UTI or other diagnostic tests that may use colorimetry to indicate a presence, amount, or concentration of a substance of interest within a sample. At block 420, the user may open a website or application on a user device, which can provide access to a system configured to implement computer vision for colorimetric testing reading.

At block 430, the user may place the reference card and the test strip in view of a camera of the user device. At block 440, the system may capture an image of the reference card and test strip and send the image to the system. At block 450, the system can receive the image and identify the reference card and test strip.

At block 460, the system can map the gradient of the reference card to a modified gradient. Such modified gradient can include a gradient that may have a higher color density than the gradient of the reference card. The modified gradient may include a single-color gradient that spans a full range of brightness values (i.e., from white or black) or a full spectrum of visible light colors (i.e., red, orange, yellow, green, blue, indigo, violet).

At block 470, the system can display the modified gradient on the user device. At block 475, the user may compare the color of the test strip to the modified gradient and select a test result number that may be associated with the color of the test strip. At block 480, the user may input the test result number to the system. The system may then direct the user accordingly, which can include a test-to-treat process that can provide the user with a path to receive medication or other treatments for the detected problem.

Alternatively, at block 490, the system may identify the color of the test strip and compare it to the modified gradient. The system may interpret the test results by selecting a test result number that may be associated with the color of the test strip. The system may then direct the user accordingly, which can include a test-to-treat process that can provide the user with a path to receive medication or other treatments for the detected problem.

FIG. 5 is a block diagram depicting an embodiment of a computer hardware system configured to run software for implementing one or more embodiments of the health testing and diagnostic systems, methods, and devices disclosed herein.

In some embodiments, the systems, processes, and methods described herein are implemented using a computing system, such as the one illustrated in FIG. 5. The example computer system 502 is in communication with one or more computing systems 520 and/or one or more data sources 522 via one or more networks 518. While FIG. 5 illustrates an embodiment of a computing system 502, it is recognized that the functionality provided for in the components and modules of computer system 502 may be combined into fewer components and modules, or further separated into additional components and modules.

The computer system 502 can comprise a module 514 that carries out the functions, methods, acts, and/or processes described herein. The module 514 is executed on the computer system 502 by a central processing unit 506 discussed further below.

In general, the word “module,” as used herein, refers to logic embodied in hardware or firmware or to a collection of software instructions having entry and exit points. Modules are written in a program language, such as Java, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 502 includes one or more processing units (CPU) 506, which may comprise a microprocessor. The computer system 502 further includes a physical memory 510, such as random-access memory (RAM) for temporary storage of information, a read-only memory (ROM) for permanent storage of information, and a mass storage device 504, such as a backing store, a hard drive, rotating magnetic disks, solid state disks (SSD), flash memory, phase-change memory (PCM), 3D XPoint memory, a diskette, or an optical media storage device. Alternatively, the mass storage device may be implemented in an array of servers. Typically, the components of the computer system 502 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 502 includes one or more input/output (I/O) devices and interfaces 512, such as a keyboard, mouse, touch pad, and printer. The I/O devices and interfaces 512 can include one or more display devices, such as a monitor, that allow the visual presentation of data to a user. More particularly, a display device provides for the presentation of graphical user interfaces (GUIs) as application software data and multimedia presentations, for example. The I/O devices and interfaces 512 can also provide a communications interface to various external devices. The computer system 502 may comprise one or more multimedia devices 508, such as speakers, video cards, graphics accelerators, and microphones, for example.

The computer system 502 may run on a variety of computing devices, such as a server, a Windows server, a Structure Query Language server, a Unix Server, a personal computer, a laptop computer, and so forth. In other embodiments, the computer system 502 may run on a cluster computer system, a mainframe computer system, and/or other computing system suitable for controlling and/or communicating with large databases, performing high volume transaction processing and generating reports from large databases. The computing system 502 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a GUI, among other things.

The computer system 502 illustrated in FIG. 5 is coupled to a network 518, such as a LAN, WAN, or the Internet via a communication link 516 (wired, wireless, or a combination thereof). Network 518 communicates with various computing devices and/or other electronic devices. Network 518 is communicating with one or more computing systems 520 and one or more data sources 522. The module 514 may access or may be accessed by computing systems 520 and/or data sources 522 through a web-enabled user access point. Connections may be a direct physical connection, a virtual connection, and other connection types. The web-enabled user access point may comprise a browser module that uses text, graphics, audio, video, and other media to present data and to allow interaction with data via the network 518.

Access to the module 514 of the computer system 502 by computing systems 520 and/or by data sources 522 may be through a web-enabled user access point such as the computing systems' 520 or data source's 522 personal computer, cell phone, smartphone, laptop, tablet computer, e-reader device, audio player, or another device capable of connecting to the network 518. Such a device may have a browser module that is implemented as a module that uses text, graphics, audio, video, and other media to present data and to allow interaction with data via the network 518.

The output module may be implemented as a combination of an all-points addressable display such as a cathode ray tube (CRT), a liquid crystal display (LCD), a plasma display, or other types and/or combinations of displays. The output module may be implemented to communicate with input devices 512 and may also include software with the appropriate interfaces that allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 502 may comprise a physical or logical connection established between a remote microprocessor and a mainframe host computer for the express purpose of uploading, downloading, or viewing interactive data and databases online in real time. The remote microprocessor may be operated by an entity operating the computer system 502, including the client server systems or the main server system, and/or may be operated by one or more of the data sources 522 and/or one or more of the computing systems 520. In some embodiments, terminal emulation software may be used on the microprocessor for participating in the micro-mainframe link.

In some embodiments, computing systems 520 that are internal to an entity operating the computer system 502 may access the module 514 internally as an application or process run by the CPU 506.

In some embodiments, one or more features of the systems, methods, and devices described herein can utilize a URL and/or cookies, for example, for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, a domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a website). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 502 may include one or more internal and/or external data sources (for example, data sources 522). In some embodiments, one or more of the data repositories and the data sources described above may be implemented using a relational database, such as DB2, Sybase, Oracle, CodeBase, and Microsoft® SQL Server, as well as other types of databases such as a flat-file database, an entity relationship database, and object-oriented database, and/or a record-based database.

The computer system 502 may also access one or more databases 522. The databases 522 may be stored in a database or data repository. The computer system 502 may access the one or more databases 522 through a network 518 or may directly access the database or data repository through I/O devices and interfaces 512. The data repository storing the one or more databases 522 may reside within the computer system 502.

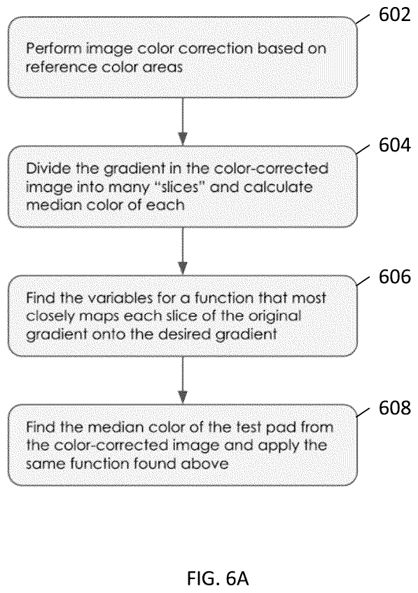

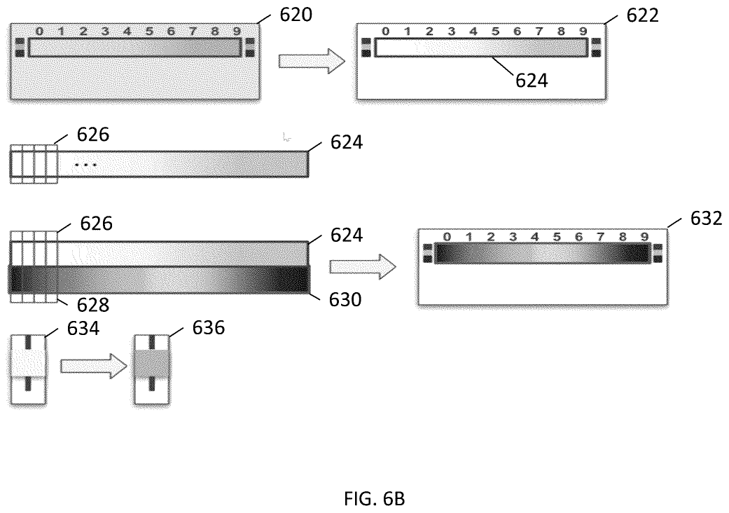

FIG. 6A is a flow diagram illustrating how color correction and gradient remapping can be performed in accordance with an example embodiment. It should be noted that various transfer functions can be utilized for the gradient remapping, and a transfer function may be specifically selected for a diagnostic test and tailored for use to remap the gradient corresponding to that particular diagnostic test.

At block 602, the system may perform an image color correction based on the reference color areas of a reference card observed in an image that includes both the reference card and a test strip. The reference color areas may include one or more reference colors that are displayed on the reference card, such as the red, green, and blue reference colors of the reference card 202 shown in FIG. 2A. In some embodiments, the values of the reference colors observed in the image may be compared to the known baseline values for those reference colors, and those differences may be used to apply color correction to the entire image. The result of this block may be a color-corrected image of the reference card and the test strip.

At block 604, the system may take the gradient on the reference card in the color-corrected image and divide the gradient into a number of slices or partitions. In some embodiments, the system may calculate the median color associated with each slice or partition of this gradient.

At block 606, the system may determine, from the color-corrected gradient, the inputs for a transfer function and apply that transfer function in order to remap the color-corrected gradient to a desired, remapped gradient. For instance, in some embodiments, the transfer function may map each slice of the color-corrected gradient into a corresponding slice of the remapped gradient. For example, the median color associated with each slice of the color-corrected gradient can be converted to corresponding value associated with the remapped gradient, and this may be performed via a lookup table that maps colors of the color-corrected gradient to colors of the remapped gradient.

At block 608, the system may determine a test color (e.g., a color from the test strip) from the color-corrected image obtained at block 602 and then apply the transfer function to this test color in order to obtain a remapped test color. In some embodiments, this step may involve finding the median color associated with the test strip from the color-corrected image before applying the transfer function. In some embodiments, the test color may be best-fit to a color from the gradient before the transfer function is applied. In some embodiments, the transfer function may determine a remapped test color from the test color via interpolation of the gradient.

FIG. 6B further illustrates how color correction and gradient remapping can be performed in accordance with FIG. 6A.

For instance, an image 620 of a reference card may be color corrected in order to obtain a color-corrected image 622 of the reference card, as indicated by block 602 of FIG. 6A. The color-corrected image 622 of the reference card may display a color-corrected gradient 624.

The color-corrected gradient 624 can be isolated and divided into many slices 626 or partitions, as indicated by block 604 of FIG. 6A. Each of the slices 626 of the color-corrected gradient 624 can be mapped to a corresponding slice 628 of the desired remapped gradient 630 based on a transfer function, as indicated by block 606 of FIG. 6B. In some embodiments, this remapped gradient 630 can then be overlaid over the image 620 of the reference card or used to generate a new image, resulting in an updated reference card 632.

Furthermore, from the color-corrected image, the test strip may be determined to have a test color 634, which can be used to obtain a remapped test color 636 by applying the transfer function to the test color 634, as indicated by block 608 of FIG. 6A. This remapped test color 636 can be displayed alongside the updated reference card 632 with the remapped gradient 630 so that the user can better discern where in the remapped gradient 630 the remapped test color 636 lies.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein, but are to be accorded the widest scope consistent with this disclosure, the principles and the novel features disclosed herein.

I/We claim:

1. A medical computer-implemented test method for improving self-diagnostic outcomes and color gradient readability during an electronic remote proctoring examination, comprising:
obtaining, by a first computing system, an image of a test strip and a reference card in a single-frame view, wherein the test strip comprises a test color associated with a result of a diagnostic test, and wherein the reference card comprises:
a gradient of colors; and
one or more reference colors;
applying, by the first computing system, a color correction to the image based on the one or more reference colors of the reference card and one or more baseline reference colors, in order to obtain a color-corrected gradient from the gradient of colors and a color-corrected test color from the test color;
remapping, by the first computing system, the color-corrected gradient into a remapped gradient based on a transfer function;
determining, by the first computing system, a remapped test color based on the color-corrected test color and the transfer function; and
generating, by the first computing system, a processed image that displays the remapped test color and the remapped gradient.

2. The method of claim 1, wherein the first computing system is a mobile smartphone.

3. The method of claim 1, wherein the diagnostic test is a medical diagnostic test.

4. The method of claim 1, wherein the remapped gradient is a high-visibility color gradient that improves a person's ability to determine where the test color lies within the gradient.

5. The method of claim 1, wherein the processed image overlays the remapped test color on the test strip.

6. The method of claim 1, wherein the processed image overlays the remapped gradient on the reference card.

7. The method of claim 1, wherein the processed image is an augmented reality image of the test strip and the reference card.

8. The method of claim 1, wherein the processed image displays the remapped test color along a length of the remapped gradient.

9. The method of claim 1, wherein the color-corrected test color is associated with a median color of the test strip.

10. The method of claim 1, wherein determining the remapped test color comprises fitting the color-corrected test color to one of the colors of the color-corrected gradient.

11. The method of claim 1, wherein the transfer function comprises:
dividing the color-corrected gradient into substantially equal partitions; and
mapping each partition of the color-corrected gradient into a corresponding partition of the remapped gradient.

12. A method comprising:
receiving, from a user device, an image of a test strip and a reference card in a single-frame view, wherein the test strip comprises a test color associated with a result of a diagnostic test, and wherein the reference card comprises:
a gradient of colors; and
one or more reference colors;
applying a color correction to the image based on the one or more reference colors of the reference card, in order to obtain a color-corrected gradient from the gradient of colors and a color-corrected test color from the test color;
remapping the color-corrected gradient into a remapped gradient based on a transfer function;
determining a remapped test color based on the color-corrected test color and the transfer function; and
transmitting the remapped test color and the remapped gradient to the user device to be displayed in a processed image.

13. The method of claim 12, wherein the diagnostic test is a medical diagnostic test.

14. The method of claim 12, wherein the remapped gradient is a high-visibility color gradient that improves a person's ability to determine where the test color lies within the gradient.

15. The method of claim 12, wherein the processed image overlays the remapped test color on the test strip.

16. The method of claim 12, wherein the processed image overlays the remapped gradient on the reference card.

17. The method of claim 12, wherein the processed image is an augmented reality image of the test strip and the reference card.

18. The method of claim 12, wherein the processed image displays the remapped test color along a length of the remapped gradient.

19. The method of claim 12, wherein the color-corrected test color is associated with a median color of the test strip.

20. The method of claim 12, wherein determining the remapped test color comprises fitting the color-corrected test color to one of the colors of the color-corrected gradient.
